(19) United States
(12) Patent Application Publication
(10) Pub. No.: US 2009/0247486 A1
(43) Pub. Date: Oct. 1, 2009

(54) COMPOSITION FOR ORAL SUBSTANCE COATING, COVERING MATERIAL FOR ORAL SUBSTANCE, EDIBLE CONTAINER AND ORAL SUBSTANCE USING THE SAME

(76) Inventor: Yuriko SERIZAWA, Ashigarakami-gun (JP)

Correspondence Address: BIRCH STEWART KOLASCH & BIRCH, PO BOX 747, FALLS CHURCH, VA, 22040-0747, US

(21) Appl. No.: 12/411,115

(22) Filed: Mar. 25, 2009

(30) Foreign Application Priority Data
Mar. 26, 2008 (JP) P2008-082215

(51) Int. Cl. A61K 31/732; A61K 31/717; A61P 43/00

(52) U.S. Cl. 514/57; 514/54

(57) ABSTRACT

A composition for oral substance coating, a covering material for an oral substance or an edible container, includes an indigestible and water-insoluble dietary fiber derived from a plant, alga or fungus, or a hemicellulose or protopectin, and an oral substance uses the same.

### BACKGROUND OF THE INVENTION

1. Field of the Invention

This invention relates to a composition which uses an indigestible and water-insoluble dietary fiber and to an oral substance that uses the same. Illustratively, it relates to a composition for oral substance coating, which coats a capsule or the like oral substance, a covering material for an oral substance such as a film or the like, which covers an oral substance, and a capsule itself or the like edible container, and more illustratively to a composition which provides large intestine disintegrating property and an oral substance that uses the same.

2. Description of the Related Art

Up to now, packing with a capsule or the like and coating have been carried out for food and medicines in order to facilitate protection and ingestion of their contents.

Regarding the medicines, studies have been carried out in recent years from the viewpoint of the drug delivery system (DDS) such as a sustained release or site-directed administration.

As a method of the site-directed administration, application of a coating which gives selective solubility to a medicine has been attempted. As the coating, for example, coatings for a sugar coating, for a film of a water-soluble polymer or the like, for a gastric juice-insoluble enteric coating and the like have been developed.

Since the large intestine is positioned at a downstream of the digestive organ system, in order to deliver an oral substance specifically at the large intestine, it is necessary that the substance is insoluble in the gastric juice and small intestinal juice and it is necessary also that it is dissolved at the large intestine, so that this purpose is difficult to achieve in comparison with the case of the specific delivery at the stomach and small intestines.

JP-B-8-13748 and Japanese Patent No. 3282832 describe pharmaceutical preparations having a large intestine disintegrating property, prepared by using a coating which is insoluble in the gastric juice and small intestinal juice but dissolved at the large intestine.

However, since the chitosan which is used in the above-mentioned coating generally uses shells of a crab and the like crustaceans as the material, persons who have crustacean allergy cannot ingest it. In addition, vitamin K absorption inhibition has been pointed out in chitosan.

### SUMMARY OF THE INVENTION

In addition to the problem of crustacean allergy, the preparation produced by the conventional chitosan coating is disintegrated at regions other than the large intestines in some cases, so that despite of the possession of large intestine disintegrating property, its further improvement is in demand.

The object of the invention is to provide a target-specific delivery measure by solving these problems. Illustratively, it is to provide compositions for oral use (a composition for oral substance coating, a covering material for an oral substance and an edible container) which protects a pharmaceutical preparation from the gastric juice and small intestinal juice, wherein the protection is relieved in the large intestine, and can be ingested even by persons who have crustacean allergy.

The present inventors have found that the above-mentioned problems are solved by a novel oral composition which comprises a dietary fiber.

That is, the invention consists of the following constructions.

(1) A composition for oral substance coating, which comprises:

an indigestible and water-insoluble dietary fiber derived from a plant, alga or fungus.

(2) A covering material for an oral substance, which comprises:

an indigestible and water-insoluble dietary fiber derived from a plant, alga or fungus.

(3) An edible container, which comprises:

an indigestible and water-insoluble dietary fiber derived from a plant, alga or fungus.

(4) A composition for oral substance coating, which comprises:

a hemicellulose or protopectin.

(5) A covering material for an oral substance, which comprises:

a hemicellulose or protopectin.

(6) An edible container, which comprises:

a hemicellulose or protopectin.

(7) A coated oral substance, which comprises:

an oral substance that is coated with the composition for oral substance coating as described in (1) or (4) above.

(8) A covered oral substance, which comprises:

an oral substance that is covered with the covering material for an oral substance as described in (2) or (5) above above.

(9) An enclosed oral substance, which comprises:

an oral substance that is enclosed in the edible container as described in (3) or (6) above above.

(10) The coated oral substance as described in (7) above, which disintegrates in a large intestine of a subject.

(11) The covered oral substance as described in (8) above, which disintegrates in a large intestine of a subject.

(12) The enclosed oral substance as described in (9) above, which disintegrates in a large intestine of a subject.

(13) The coated oral substance as described in (7) or (10) above, which is a medicine or food.

(14) The covered oral substance as described in (8) or (11) above, which is a medicine or food.

(15) The enclosed oral substance as described in (9) or (12) above, which is a medicine or food.

(16) A method for delivering an oral substance specifically to a large intestine of a subject, the oral substance containing a material having an ability to increase an effect of the material when released in the large intestine of the subject, the method comprising:

coating the oral substance with the composition for oral substance coating as described in (1) or (4) above.

(17) A method for delivering an oral substance specifically to a large intestine of a subject, the oral substance containing a material having an ability to increase an effect of the material when released in the large intestine of the subject, the method comprising:

covering the oral substance with the covering material for an oral substance as described in (2) or (5) above.

(18) A method for delivering an oral substance specifically to a large intestine of a subject, the oral substance containing a material having an ability to increase an effect of the material when released in the large intestine of the subject, the method comprising:

enclosing the oral substance in the edible container as described in (3) or (6) above.

### DETAILED DESCRIPTION OF THE INVENTION

<Dietary Fiber>

The oral compositions of the invention (composition for oral substance coating, covering material for an oral substance and edible container) comprise a dietary fiber.

The dietary fiber is preferably a substance derived from a plant, alga or fungus. By the use of a dietary fiber derived from a plant, alga or fungus, a coating which causes relatively lesser allergy and is safe for oral ingestion can be provided.

The dietary fiber of the invention is indigestible and insoluble in water. The term “digestion” as used herein is dissolution by the gastric juice and small intestinal juice. The term “indigestible” according to the invention illustratively means that there is no solubility in the first liquid and second liquid in accordance with the “disintegration test” of The Pharmacopoeia of Japan, 15^{th} revision, (general test, 6.09). Also, the term “insoluble in water” illustratively means that there is no solubility in water in accordance with the “disintegration test” of The Pharmacopoeia of Japan, 15^{th} revision, (general test, 6.09). The “disintegration test” of The Pharmacopoeia of Japan, 15^{th} revision, (general test, 6.09) mentioned above is herein incorporated by reference.

In this connection, the term “indigestible and insoluble in water” is a property of the dietary fiber itself, and in the case of glucomannan for example, it becomes insoluble in water when treated with an alkali but the dietary fiber itself is soluble in water so that it is not included in the dietary fiber of this application which is indigestible and insoluble in water.

It is desirable that the composition for oral substance coating, covering material for an oral substance, edible container and oral substance using them of the invention are dissolved at inside of the large intestine. That is, it is desirable that the composition for oral substance coating, the covering material for an oral substance, the edible container and the oral substance coated with the composition for oral substance coating, covered with the covering material for an oral substance or enclosed in the edible container of the invention have a large intestine disintegrating property.

The composition for oral substance coating, the covering material for an oral substance, the edible container and the oral substance coated with the composition for oral substance coating, covered with the covering material for an oral substance or enclosed in the edible container of the invention are not limited as long as they are dissolved at inside of the large intestine, but it is desirable that they are dissolved at inside of the large intestine of human. That is, it is desirable that the composition for oral substance coating, the covering material for an oral substance, the edible container and the oral substance coated with the composition for oral substance coating, covered with the covering material for an oral substance or enclosed in the edible container of the invention have a human large intestine disintegrating property.

Hemicellulose or Protopectins

The dietary fiber of the invention is preferably hemicellulose or protopectins.

The hemicellulose is preferably xylan, glucuronoxylan, arabinoxylan, arabinan, arabinogalactan, xyloglucan, 1,3-1,4-β-D-glucan, callose, glucuronoarabinoxylan or methylglucuronoxylan. More preferred is xylan.

The hemicellulose and protopectin are indigestible and insoluble in water by themselves and degraded by the enzymes possessed by bacteria in the large intestine.

Accordingly, an oral substance on which coating with a hemicellulose or protopectin or a derivative thereof is carried out passes through the stomach and small intestines as being protected by the coating, and the coating is relieved at inside of the large intestine. Because of this, it becomes possible to deliver the oral substance specifically to the large intestine.

The hemicellulose or protopectin and derivatives thereof to be used in the invention may be synthesized substances, but these can also be obtained from a plant, alga or fungus.

Hemicellulose is a polysaccharide obtained by extracting a dietary fiber with an alkali, which is a component that constitutes the plant cell wall together with cellulose and lignin and is insoluble in water.

Hemicellulose can be extracted by carrying out a blasting, a high pressure cooking treatment or an alkali solution (potassium hydroxide, sodium hydroxide or sodium acid carbonate) treatment of, for example, the insoluble components of oats, corn, bamboo grass, white birch, sugar cane, chaff, peanut shell, bagasse, thinnings, cotton seed meal and the like.

Also, protopectin is an insoluble component of apple, strawberry and the like and is extracted for example by dissolving it in an acidic solution.

These may be extracted from a plant, alga or fungus by a general method, or various commercially available articles may be used. In addition, those which are chemically synthesized may also be used.

Other Components

In addition to the above-mentioned alga- or fungus-derived dietary fiber or a hemicellulose or protopectin, the composition for oral substance coating, covering material for an oral substance or edible container of the invention can contain a lubricant, a saccharide, a coloring agent, a disintegrating agent, a binder and the like additive components which are acceptable as oral substances. As such substances, those which are used in general food, quasi drugs or medicines can be optionally used.

According to the oral composition of the invention, containing ratio of the alga- or fungus-derived dietary fiber or a hemicellulose or protopectin with other solid components (total) is from 100:0 to 30:70, more preferably from 100:0 to 50:50, further preferably from 100:0 to 70:30, as mass ratio.

Solvent

According to the composition for oral substance coating of the invention, it is desirable that the above-mentioned alga- or fungus-derived dietary fiber or a hemicellulose or protopectin and other containable solid components are dissolved in a solvent.

Regarding the solvent, kind and concentration of various solvents generally used in the coating of food and medicines, such as water, an alcohol or the like organic solvent, a hydrochloric acid or sodium hydroxide solution or the like inorganic solvent, can be optionally selected in response to the coating method.

In the case of hemicellulose, a sodium acid carbonate solution, a sodium hydroxide solution or a potassium hydroxide solution is desirable, and in the case of protopectin, hydrochloric acid, a citric acid solution or acetic acid is desirable.

When the oral composition of the invention is a solution, its solid substance concentration is preferably from 1% by mass to 75% by mass, more preferably from 5 to 60% by mass, particularly preferably from 5 to 40% by mass. (In this specification, mass ratio is equal to weight ratio.)

Covering Material for an Oral Substance

The oral composition of the invention which comprises an alga- or fungus-derived dietary fiber or a hemicellulose or protopectin may be provided by making into a covering material for an oral substance in the form of a solvent-free film. As the film, those which have a film thickness of approximately from 5 to 500 μm are suitable. When provided as a film, each user can wrap up food, medicine and the like. The film is formed by a general method.

Edible Container

The oral composition of the invention maybe a capsule or the like edible container. That is, it is preferably a capsule which comprises an alga- or fungus-derived dietary fiber or a hemicellulose or protopectin.

The capsule means a hollow oral container capable of containing a medicine or functional food, and its examples include generally used empty capsules and the like. Illustratively, those which are established by The Pharmacopoeia of Japan, 15^{th} revision, can be exemplified, but according to the invention, it may be in a minute form such as the so-called microcapsule.

<Oral Substance which Uses the Oral Composition>

“An oral substance” in this specification means a substance that is ingested from a mouth and delivered to the digestive tract.

The oral substance which uses the oral composition of the invention may be any one of food, medicines and quasi drugs.

The oral substance which uses the oral composition of the invention is basically a solid matter. Illustratively, it is a pharmaceutical preparation, a capsule or a food.

The oral substance which uses the oral composition of the invention may be those in which the oral composition itself is a container of a drug and the like contents as described in the foregoing or those in which a general pharmaceutical preparation or the like is coated with the oral composition of the invention. Preferred is an oral substance coated with the composition for oral substance coating of the invention (also referred to as coated oral substance), an oral substance covered with the covering material for an oral substance of the invention (also referred to as covered oral substance) or an oral substance enclosed in the edible container of the invention (also referred to as enclosed oral substance).

In this case, illustrative examples of the pharmaceutical preparation include granules, tablets, pills, powders and capsules in which a drug is enclosed in capsules, and characteristics of the respective pharmaceutical preparations are as established by The Pharmacopoeia of Japan, 15^{th} revision.

Regarding the pharmaceutical preparation, usual various pharmaceutical preparations which can contain a drug to be used as the active ingredient and a filer such as lactose, starch or the like, a binder, a disintegrating agent, a lubricant and the like can become the coating objects in the invention.

The following illustratively describes on the capsule.

As the capsule, those which have hard capsule, soft capsule, microcapsule, seamless capsule and the like various shapes can be exemplified.

The capsule may have a coat of gelatin, a hydrophilic polymer or the like.

It is desirable that the hydrophilic polymer is a hydrophilic polymer obtained by purifying or synthesizing using a natural animal or plant or the like as the origin, or a processed polymer thereof, and at least one species selected from alginic acid or a salt thereof, agar gum, guar gum, locust bean gum, tara gum, ghatti gum, Carya glandifolia gum, tragacanth gum, karaya gum, pectin, gum arabic, xanthan gum, gellan gum, starch, konjak mannan, galactomannan, funoran, acetan rubber, welan, rhamsan, furcelan, succinoglycan, sclenoglycan, schizophyllan, tamarind gum, curdlan, carrageenan, pullulan and dextran can be cited as its examples. These may be used as a combination of two or more species or can also be combined with the above-mentioned pig skin gelatin. These hydrophilic polymers may be those in which natural products are processed. Particularly desirable among them are pullulan, carrageenan and dextran and especially desirable is carrageenan.

As the gelatin, a hot water-extracted protein of proteins obtained by using various animals, fishes and the like as the material can be cited. Illustratively, it can be purified via an ion exchange treatment step after treatment of these living beings with an acid or alkali and subsequent extraction by heating in water.

Since molecular weight of the gelatin or natural hydrophilic polymer of the invention can be reduced by an enzyme treatment and the like, its average molecular weight can be optionally selected, which is generally from 1 to 5,000,000, preferably from 10,000 to 5,000,000, more preferably from 10,000 to 2,500,000, further preferably from 10,000 to 1,000,000, particularly preferably from about 10,000 to 500,000.

The capsule coat may further contain oil and fat, a polyhydric alcohol, a surfactant, an antioxidant, a coloring agent, an aromatic and the like.

As the oil and fat, for example, evening prime rose oil, soybean oil, safflower oil, olive oil, germ oil, rapeseed oil, sunflower oil, peanut oil, cotton seed oil, rice bran oil, cocoa butter and the like natural oils and hydrogenated oils thereof, fatty acid glycerides (glyceride, diglyceride, triglyceride and the like) and the like can be exemplified, and polyethylene glycol, propylene glycol, glycerol, sorbitol and the like as the polyhydric alcohol, a sorbitan fatty acid ester, a polyglycerol fatty acid ester and the like nonionic surfactants as the surfactant, and a carotenoid system pigment, an anthocyanin system pigment, a cacao pigment, an anthranon system pigment, a caramel pigment and the like as the pigment. Particularly, addition of oil and fat, a polyhydric alcohol, a surfactant and a natural pigment to the capsule coat is suitable from the viewpoint that stabilization of pharmaceutical capsule preparations can be further improved.

The oral substance which uses the oral composition of the invention preferably contains therein a material having the ability to increase its effect when released in the large intestine.

As such a material, for example, those which are useful for improving environment of enteric bacteria, such as lactic acid bacteria, saccharides (glucose and the like monosaccharides, sucrose and the like disaccharides, oligosaccharides and the like), antibiotics, antiviral agents and the like, those which can directly act on the large intestinal wall, such as anti-diarrheal drugs, anti-constipation drugs, remedies for Crohn disease, remedies for irritable bowel syndrome, remedies for ulcerative colitis, antitumor agents and the like, and the like can be cited, and more preferably, a lactic acid bacterium, a bifidobacterium, glucose, sucrose, an oligosaccharide, a saccharification bacterium, a butyric acid bacterium, lactoferrin and a polysaccharide can be cited.

<Coating>

Regarding the method for coating the composition for oral substance coating on an oral substance, those which are similar to the methods conventionally used for oral substances including the methods for completing by a film or taping, such as spraying, pan coating, fluidized bed coating, compression coating and the like, can be exemplified.

Thickness of the coating is preferably from 5 to 1,000 μm, more preferably from 10 to 700 μm, particularly preferably from 30 to 300 μm.

### EXAMPLES

The following describes the invention using examples, but the invention is not limited to the following examples.

In this connection, Xylan Oat Spelts manufactured by Wako Pure Chemical Industries, Ltd. was used as the xylan in the examples.

Also, the protopectin was extracted based on the method of T. Sakai, M. Okushima; Purification and Crystallization of a Protopectin-solubilizing Enzyme from Trichosporon penicillatum, Agric. Biol. Chem., 46(3), 667-676 (1982).

### Production Example 1

A uniform liquid was prepared by dissolving 10% by mass of protopectin in 5% by mass in concentration of acetic acid aqueous solution or 10% by mass of lactic acid aqueous solution. A tablet or capsule was spray-coated with this solution to form a coat of 0.05 g per 1 g of the pharmaceutical preparation, soaked in 10% sodium hydroxide aqueous solution for 5 minutes, washed by further soaking it in water for 30 minutes and then dried.

Next, the tablet or capsule was coated with alcohol-dissolved shellac (BS 30, Gifu Shellac Manufacturing Co., Ltd.). The coating was carried out by applying film coating using HICOATER in the case of the tablet or by soaking in a shellac solution in the case of the capsule. A gastric acid-resistant coating treatment was carried out by spraying an alcohol solution of 10% by mass of shellac based on the tablet mass. A 0.075 g per 1 g pharmaceutical preparation of shellac coating was applied. After completion of the coating treatment, a coated tablet or capsule was obtained by drying it for 24 hours.

### Production Example 2

A uniform liquid was prepared by dissolving 10% by mass of xylan in 10% by mass of sodium acid carbonate solution. A tablet or capsule was spray-coated with this solution to form a coat of 0.05 g per 1 g pharmaceutical preparation, soaked in 10% hydrochloric acid for 5 minutes, washed by further soaking it in water for 30 minutes and then dried.

Next, the tablet or capsule was coated with alcohol-dissolved shellac (BS 30, Gifu Shellac Manufacturing Co., Ltd.). The coating was carried out by applying film coating using HICOATER in the case of the tablet or by soaking in a shellac solution in the case of the capsule. A gastric acid-resistant coating treatment was carried out by spraying an alcohol solution of 10% by mass of shellac-based on the tablet mass. A 0.075 g per 1 g pharmaceutical preparation of shellac coating was applied. After completion of the coating treatment, a coated tablet or capsule was obtained by drying it for 24 hours.

### Production Example 3

A uniform liquid was prepared by dissolving 10% by mass of arabinoxylan in 10% by mass of sodium acid carbonate solution. A tablet or capsule was spray-coated with this solution to form a coat of 0.05 g per 1 g pharmaceutical preparation, soaked in 10% of hydrochloric acid for 5 minutes, washed by further soaking it in water for 30 minutes and then dried.

Next, the tablet or capsule was coated with alcohol-dissolved shellac (BS 30, Gifu Shellac Manufacturing Co., Ltd.). The coating was carried out by applying film coating using HICOATER in the case of the tablet or by soaking in a shellac solution in the case of the capsule. A gastric acid-resistant coating treatment was carried out by spraying an alcohol solution of 10% by mass of shellac based on the tablet mass. A 0.075 g per 1 g pharmaceutical preparation of shellac coating was applied. After completion of the coating treatment, a coated tablet or capsule was obtained by drying it for 24 hours.

### Production Example 4

A uniform liquid was prepared by dissolving 10% by mass of protopectin in 5% by mass in concentration of acetic acid aqueous solution or 10% by mass of lactic acid aqueous solution. A cylindrical mold with its tip having a capsular shape was soaked in this solution, pulled up at a predetermined rate, soaked in 10% sodium hydroxide aqueous solution for 2 minutes, washed by further soaking it in water for 30 minutes and then dried at 40° C. in an oven for 4 hours. After the hot air drying, the capsule was pulled out from the mold and cut into a desired size to form a capsule of protopectin. The main body of the capsule was 6 mm in inner diameter×150 μm in coating thickness, and the cap part was 6.2 mm in inner diameter×150 μm in coating thickness.

### Production Example 5

A uniform liquid was prepared by dissolving 10% by mass of xylan in 10% by mass of sodium acid carbonate aqueous solution. A cylindrical mold with its tip having a capsular shape was soaked in this solution, pulled up at a predetermined rate, soaked in 10% hydrochloric acid aqueous solution for 2 minutes, washed by further soaking it in water for 30 minutes and then dried at 40° C. in an oven for 4 hours. After the hot air drying, the capsule was pulled out from the mold and cut into a desired size to form a capsule of xylan. The main body of the capsule was 6 mm in inner diameter×150 μm in coating thickness, and the cap part was 6.2 mm in inner diameter×150 μm in coating thickness.

### Production Example 6

A uniform liquid was prepared by dissolving 10% by mass of arabinoxylan in 10% by mass of sodium acid carbonate aqueous solution. A cylindrical mold with its tip having a capsular shape was soaked in this solution, pulled up at a predetermined rate, soaked in 10% hydrochloric acid aqueous solution for 2 minutes, washed by further soaking it in water for 30 minutes and then dried at 40° C. in an oven for 4 hours. After the hot air drying, the capsule was pulled out from the mold and cut into a desired size to form a capsule of arabinoxylan. The main body of the capsule was 6 mm in inner diameter×150 μm in coating thickness, and the cap part was 6.2 mm in inner diameter×150 μm in coating thickness.

### Test Example 1

Regarding the protopectin coating capsule, xylan coating capsule and arabinoxylan coating capsule prepared by the methods of Production Examples 1 to 3 and the protopectin capsule, xylan capsule and arabinoxylan capsule prepared by the methods of Production Examples 4 to 6, wherein a food dye Red No. 106 and crystalline cellulose were mixed and enclosed in a gelatin capsule (Capsugel Japan Inc.), a disintegration test was carried out in accordance with The Pharmacopoeia of Japan, 15^{th} revision, using those in which the food dye Red No. 106 was filled in each capsule and the junction of the body and cap was sealed with the protopectin solution, xylan solution or arabinoxylan solution used in the capsule forming and then dried. The test was carried out firstly using the disintegration test first liquid for 2 hours and then using the second liquid for 4 hours at the longest. In addition, a dissolution test for water was carried out in the same manner for 6 hours.

TABLE 1
The Pharmacopoeia of Japan Disintegration test (capsules)

| | | First liquid | Second liquid | Water |
|---|---|---|---|---|
| Production | Protopectin | Not | Not | Not |
| Example 1 | coating capsule | disintegrated | disintegrated | disintegrated |
| Production | Xylan coating | Not | Not | Not |
| Example 2 | capsule | disintegrated | disintegrated | disintegrated |
| Production | Arabinoxylan | Not | Not | Not |
| Example 3 | coating capsule | disintegrated | disintegrated | disintegrated |
| Production | Protopectin | Not | Not | Not |
| Example 4 | capsule | disintegrated | disintegrated | disintegrated |
| Production | Xylan capsule | Not | Not | Not |
| Example 5 | | disintegrated | disintegrated | disintegrated |
| Production | Arabinoxylan | Not | Not | Not |
| Example 6 | capsule | disintegrated | disintegrated | disintegrated |

From the above results, it was revealed that the preparations which used the coating of the invention do not dissolve in water and disintegrate at the stomach and small intestines.

### Test Example 2

A disintegration test was carried out in accordance with The Pharmacopoeia of Japan, 15^{th} revision, on the tablets prepared by subjecting a mixture of a food dye Red No. 106 and crystalline cellulose to a tablet machine and applying a coating thereto by the above-mentioned method. The test was carried out firstly using the disintegration test first liquid for 2 hours and then using the second liquid for 4 hours at the longest. In addition, a dissolution test for water was carried out in the same manner for 6 hours.

TABLE 2
The Pharmacopoeia of Japan Disintegration test (tablets)

| | | First liquid | Second liquid | Water |
|---|---|---|---|---|
| Production | Protopectin | Not | Not | Not |
| Example 1 | coating tablet | disintegrated | disintegrated | disintegrated |
| Production | Xylan coating | Not | Not | Not |
| Example 2 | tablet | disintegrated | disintegrated | disintegrated |
| Production | Arabinoxylan | Not | Not | Not |
| Example 3 | coating tablet | disintegrated | disintegrated | disintegrated |

From the above results, it was revealed that the preparations which used the coating of the invention do not dissolve in water and disintegrate at the stomach and small intestines.

### Test Example 3

In order to verify the large intestine disintegrating ability by the above-mentioned coating, an intestinal dominant species, Bacteroides fragilis ATCC 25285, was liquid-cultured, and a sample in which a red dye, Congo Red, was filled in the xylan capsule prepared by the method of Production Example 4 and the junction of the body and cap was sealed with the xylan solution used in the capsule forming and then dried was put into this culture medium and shaken at 37° C. for 9 hours under an anaerobic condition. As a result, elution of the dye into the culture medium was found after 3 hours and disintegration of the capsule was confirmed after 6 hours. In addition, since disintegration of the capsule was not found even after 9 hours in the case of the culture medium used as a control to which the bacterium was not added, it was revealed that the xylan capsule is disintegrated by a large intestinal bacterium.

### Test Example 4

In order to verify the large intestine disintegrating ability by the above-mentioned coating, an intestinal dominant species, B. fragilis ATCC 25285, was liquid-cultured, and a xylan coating tablet prepared by mixing the red dye Congo Red and crystalline cellulose, making the mixture into a tablet and coating it by the method of Production Example 2 was shaken in this culture medium at 37° C. for 9 hours under an anaerobic condition. As a result, elution of the dye into the culture medium was found after 3 hours and disintegration of the tablet was confirmed after 4.5 hours. In addition, since disintegration of the tablet was not found even after 9 hours in the case of the culture medium used as a control to which the bacterium was not added, it was revealed that the xylan coating tablet is disintegrated by a large intestinal bacterium.

### Production Example 7

A 100 mg portion of a lactic acid bacterium Lactobacillus acidophilus (Morinaga Milk Industry Co., Ltd.), 20 mg of Morinaga Bifidobacterium Powder BB 536 and 130 mg of crystalline cellulose were mixed and enclosed in a swine-derived gelatin capsule (Capsugel Japan Inc.), and the thus obtained capsule was subjected to the coating by the method of Production Example 1, 2 or 3.

### Production Example 8

A 100 mg portion of the lactic acid bacterium L. acidophilus (Morinaga Milk Industry Co., Ltd.), 50 mg of the bifidobacterium Morinaga Bifidobacterium Powder BB 536 and 150 mg of crystalline cellulose were mixed and subjected to tablet making using a portable simplified tablet molding machine HANDTAB-100 (mfd. by ICHIHASHI SEIKI), and the thus obtained tablet was coated by the method of Production Example 1, 2 or 3.

### Production Example 9

A gastric acid-resistant coating treatment of capsules prepared by the method of Production Examples 7 and 8 was carried out by spraying thereon an alcohol solution of 10% by mass of shellac based on the capsule mass.

By the use of the oral composition of the invention in an oral substance, a target-specific drug delivery measure which can be ingested even by persons who have crustacean allergy can be provided. Illustratively, there is provided a coating which protects a pharmaceutical preparation from the gastric juice and small intestinal juice, wherein the protection is relieved in the large intestine, and can be ingested even by persons who have crustacean allergy.

The entire disclosure of each and every foreign patent application from which the benefit of foreign priority has been claimed in the present application is incorporated herein by reference, as if fully set forth.

What is claimed is:

1. A composition for oral substance coating, which comprises:
an indigestible and water-insoluble dietary fiber derived from a plant, alga or fungus.

2. A covering material for an oral substance, which comprises:
an indigestible and water-insoluble dietary fiber derived from a plant, alga or fungus.

3. An edible container, which comprises:
an indigestible and water-insoluble dietary fiber derived from a plant, alga or fungus.

4. A composition for oral substance coating, which comprises:
a hemicellulose or protopectin.

5. A covering material for an oral substance, which comprises:
a hemicellulose or protopectin.

6. An edible container, which comprises:
a hemicellulose or protopectin.

7. A coated oral substance, which comprises:
an oral substance that is coated with the composition for oral substance coating according to claim 1.

8. A covered oral substance, which comprises:
an oral substance that is covered with the covering material for an oral substance according to claim 2.

9. An enclosed oral substance, which comprises:
an oral substance that is enclosed in the edible container according to claim 3.

10. The coated oral substance according to claim 7, which disintegrates in a large intestine of a subject.

11. The covered oral substance according to claim 8, which disintegrates in a large intestine of a subject.

12. The enclosed oral substance according to claim 9, which disintegrates in a large intestine of a subject.

13. The coated oral substance according to claim 7, which is a medicine or food.

14. The covered oral substance according to claim 8, which is a medicine or food.

15. The enclosed oral substance according to claim 9, which is a medicine or food.

16. A method for delivering an oral substance specifically to a large intestine of a subject, the oral substance containing a material having an ability to increase an effect of the material when released in the large intestine of the subject, the method comprising:
coating the oral substance with the composition for oral substance coating according to claim 1.

17. A method for delivering an oral substance specifically to a large intestine of a subject, the oral substance containing a material having an ability to increase an effect of the material when released in the large intestine of the subject, the method comprising:
covering the oral substance with the covering material for an oral substance according to claim 2.

18. A method for delivering an oral substance specifically to a large intestine of a subject, the oral substance containing a material having an ability to increase an effect of the material when released in the large intestine of the subject, the method comprising:
enclosing the oral substance in the edible container according to claim 3.
